(19) United States
(12) Patent Application Publication
(10) Pub. No.: US 2018/0027090 A1
(43) Pub. Date: Jan. 25, 2018

(54) INFORMATION PROCESSING DEVICE, INFORMATION PROCESSING METHOD, AND PROGRAM

(71) Applicant: SONY CORPORATION, Tokyo (JP)

(72) Inventors: Yoshihiro NAKANISHI, Tokyo (JP); Ryo MUKAIYAMA, Tokyo (JP); Hideyuki MATSUNAGA, Kanagawa (JP)

(21) Appl. No.: 15/548,331

(22) PCT Filed: Dec. 17, 2015
(86) PCT No.: PCT/JP2015/085377

§ 371 Date: Aug. 2, 2017

(30) Foreign Application Priority Data
Feb. 23, 2015 (JP) 2015-033055

(51) Int. Cl. H04L 29/08; A61B 5/04; H04W 4/02; G06F 17/30

(52) U.S. Cl. CPC H04L 67/306; G06F 17/30766; G06F 17/30764; A61B 5/04; H04W 4/02; H04W 88/02

(57) ABSTRACT

[Object] To propose an information processing device, an information processing method, and a program, each of which is capable of extracting appropriate content in accordance with a state of a user. [Solution] There is provided an information processing device including: a context information acquisition unit configured to acquire context information on a state of a user obtained by analyzing information including at least one piece of sensing data regarding the user; and a content extraction unit configured to extract one or more pieces of content from a content group on the basis of the context information.

### TECHNICAL FIELD

The present disclosure relates to an information processing device, an information processing method, and a program.

### BACKGROUND ART

In recent years, an enormous amount of content such as a text file, a still image file, a moving image file, and an audio file has been accumulated. Conventionally, in order that a user views those pieces of content, the user inputs a keyword related to a piece of content that the user desires to view and extracts a desired piece of content on the basis of the input keyword as disclosed in, for example, Patent Literature 1.

### CITATION LIST

### Patent Literature

Patent Literature 1: JP 2013-21588A

### DISCLOSURE OF INVENTION

### Technical Problem

However, in, for example, a technology disclosed in Patent Literature 1, content appropriate for a user is not extracted in some cases. For example, in order to extract content based on a mental state of the user, it cannot be said that extraction of content using a keyword is an optimal method because it is difficult to express the mental state in an appropriate keyword.

In view of the above circumstances, the present disclosure proposes an information processing device, an information processing method, and a program, each of which is new, is improved, and is capable of extracting appropriate content in accordance with a state of a user.

### Solution to Problem

According to the present disclosure, there is provided an information processing device including: a context information acquisition unit configured to acquire context information on a state of a user obtained by analyzing information including at least one piece of sensing data regarding the user; and a content extraction unit configured to extract one or more pieces of content from a content group on the basis of the context information.

Further, according to the present disclosure, there is provided an information processing method including: acquiring context information on a state of a user obtained by analyzing information including at least one piece of sensing data regarding the user; and causing a processor to extract one or more pieces of content from a content group on the basis of the context information.

Further, according to the present disclosure, there is provided a program for causing a computer to realize a function of acquiring context information on a state of a user obtained by analyzing information including at least one piece of sensing data regarding the user, and a function of extracting one or more pieces of content from a content group on the basis of the context information.

### Advantageous Effects of Invention

As described above, according to the present disclosure, it is possible to extract appropriate content in accordance with a state of a user.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is a system chart showing a configuration of a system according to first and second embodiments of the present disclosure.

FIG. 2 shows a functional configuration of a detection device according to the first and second embodiments of the present disclosure.

FIG. 3 shows a functional configuration of a server according to the first embodiment of the present disclosure.

FIG. 4 shows a functional configuration of a terminal device according to the first and second embodiments of the present disclosure.

FIG. 5 shows a sequence of information processing according to the first embodiment of the present disclosure.

FIG. 6 is a first explanatory view for describing a first example.

FIG. 7 is a second explanatory view for describing the first example.

FIG. 8 is an explanatory view for describing a second example.

FIG. 9 is a first explanatory view for describing a third example.

FIG. 10 is a second explanatory view for describing the third example.

FIG. 11 is an explanatory view for describing a fourth example.

FIG. 12 shows a functional configuration of a server according to the second embodiment of the present disclosure.

FIG. 13 shows a sequence of information processing according to the second embodiment of the present disclosure.

FIG. 14 is an explanatory view for describing a fifth example.

FIG. 15 is a block diagram showing a configuration of an information processing device according to the first and second embodiments of the present disclosure.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

Note that description will be provided in the following order.

### 1. First Embodiment

1-1. Configuration of system
1-2. Functional configuration of detection device
1-3. Functional configuration of server
1-4. Functional configuration of terminal device
2. Information processing method
2-1. First example
2-2. Second example
2-3. Third example
2-4. Fourth example

### 3. Second Embodiment

3-1. Functional configuration of server
3-2. Information processing method
3-3. Fifth example
4. Hardware configuration

### 5. Supplement

### 1. First Embodiment

Hereinafter, a first embodiment of the present disclosure will be described. First, schematic functional configurations of a system and each device according to the first embodiment of the present disclosure will be described with reference to the drawings.

### 1-1. Configuration of System

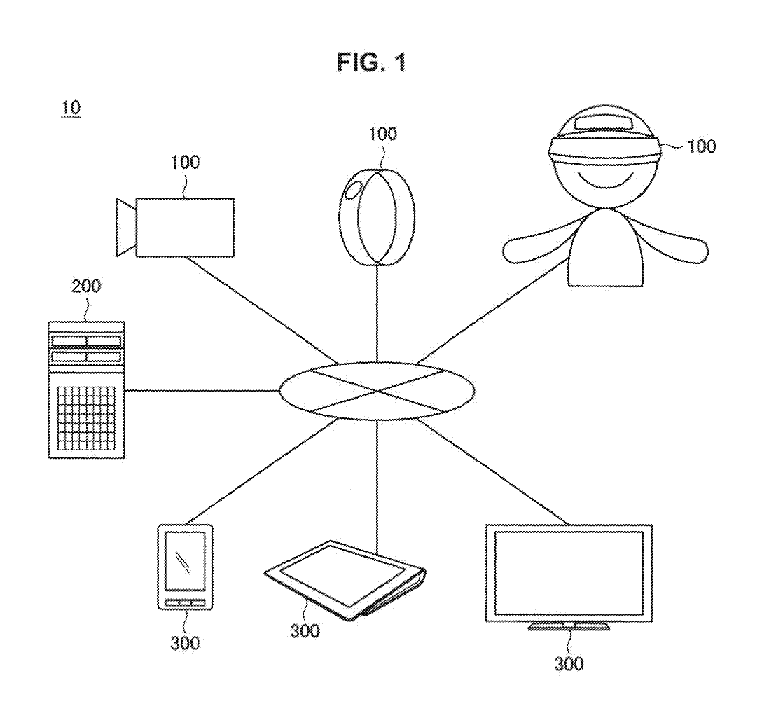

FIG. 1 is a system chart showing a schematic configuration of the system according to the first embodiment of the present disclosure. When referring to FIG. 1, a system 10 can include detection devices 100, a server 200, and terminal devices 300. The detection devices 100, the server 200, and the terminal devices 300 described above can communicate with one another via various wired or wireless networks. Note that the numbers of the detection devices 100 and the terminal devices 300 included in the system 10 are not limited to the numbers thereof shown in FIG. 1 and may be larger or smaller.

The detection device 100 detects one or more states of a user and transmits sensing data regarding the detected state(s) of the user to the server 200.

The server 200 acquires the sensing data transmitted from the detection device 100, analyzes the acquired sensing data, and acquires context information indicating the state(s) of the user. Furthermore, the server 200 extracts one or more pieces of content from a content group that can be acquired via a network on the basis of the acquired context information. Further, the server 200 can also transmit content information on the extracted one or more pieces of content (title, storage location, content, format, capacity, and the like of content) to the terminal device 300 and the like.

The terminal device 300 can output the content information transmitted from the server 200 to the user.

All the detection devices 100, the server 200, and the terminal devices 300 described above can be realized by, for example, a hardware configuration of an information processing device described below. In this case, each device does not necessarily need to be realized by a single information processing device and may be realized by, for example, a plurality of information processing devices that are connected via various wired or wireless networks and cooperate with each other.

### 1-2. Functional Configuration of Detection Device

The detection device 100 may be, for example, a wearable device worn on a part of a body of a user, such as eyewear, wristwear, or a ring-type terminal. Alternatively, the detection device 100 may be, for example, an independent camera or microphone that is fixed and placed. Furthermore, the detection device 100 may be included in a device carried by the user, such as a mobile phone (including a smartphone), a tablet-type or notebook-type personal computer (PC), a portable media player, or a portable game console. Further, the detection device 100 may be included in a device placed around the user, such as a desktop-type PC or TV, a stationary media player, a stationary game console, or a stationary telephone. Note that the detection device 100 does not necessarily need to be included in a terminal device.

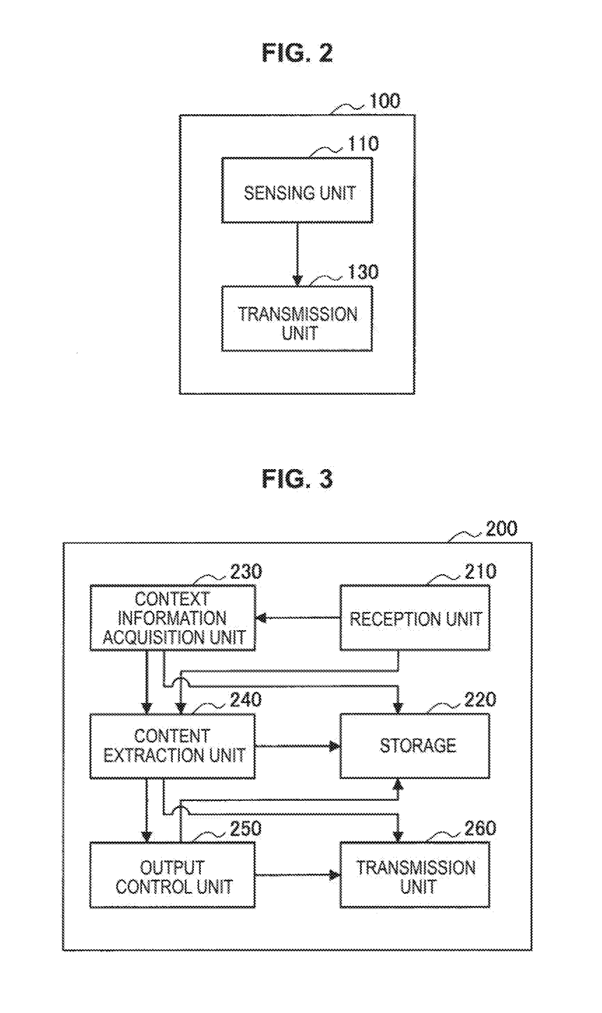

FIG. 2 shows a schematic functional configuration of the detection device 100 according to the first embodiment of the present disclosure. As shown in FIG. 2, the detection device 100 includes a sensing unit 110 and a transmission unit 130.

The sensing unit 110 includes at least one sensor for providing sensing data regarding the user. The sensing unit 110 outputs generated sensing data to the transmission unit 130, and the transmission unit 130 transmits the sensing data to the server 200. Specifically, for example, the sensing unit 110 can include a motion sensor for detecting movement of the user, a sound sensor for detecting sound generated around the user, and a biosensor for detecting biological information of the user. Furthermore, the sensing unit 110 can include a position sensor for detecting position information of the user. For example, in a case where a plurality of sensors are included, the sensing unit 110 may be separated into a plurality of parts.

Herein, the motion sensor is a sensor for detecting movement of the user and can specifically include an acceleration sensor and a gyro sensor. Specifically, the motion sensor detects a change in acceleration, an angular velocity, and the like generated in accordance with movement of the user and generates sensing data indicating those detected changes.

The sound sensor can specifically be a sound collection device such as a microphone. The sound sensor can detect not only sound uttered by the user (not only an utterance but also production of sound that does not particularly make sense, such as onomatopoeia or exclamation, may be included) but also sound generated by movement of the user, such as clapping hands, environmental sound around the user, an utterance of a person positioning around the user, and the like. Furthermore, the sound sensor may be optimized to detect a single kind of sound among the kinds of sound exemplified above or may be configured so that a plurality of kinds of sound can be detected.

The biosensor is a sensor for detecting biological information of the user and can include, for example, a sensor that is directly worn on a part of the body of the user and measures a heart rate, a blood pressure, a brain wave, respiration, perspiration, a muscle potential, a skin temperature, an electric resistance of skin, and the like. Further, the biosensor may include an imaging device and detect eye movement, a size of a pupil diameter, a gaze time, and the like.

The position sensor is a sensor for detecting a position of the user or the like and can specifically be a global navigation satellite system (GNSS) receiver or the like. In this case, the position sensor generates sensing data indicating latitude/longitude of a current position on the basis of a signal from a GNSS satellite. Further, it is possible to detect a relative positional relationship of the user on the basis of, for example, information of radio frequency identification (RFID), an access point of Wi-Fi, or a wireless base station, and therefore it is also possible to use those communication devices as a position sensor. Further, a receiver for receiving a wireless signal of Bluetooth (registered trademark) or the like from the terminal device 300 existing around the user can also be used as a position sensor for detecting a relative positional relationship with the terminal device 300.

Further, the sensing unit 110 may include an imaging device for capturing an image of the user or the user's surroundings by using various members such as an imaging element and a lens for controlling image formation of a subject image on the imaging element. In this case, for example, movement of the user is captured in an image captured by the imaging device.

The sensing unit 110 can include not only the above sensors but also various sensors such as a temperature sensor for measuring an environmental temperature.

Furthermore, the detection device 100 may include a reception unit (not shown) for acquiring information such as control information for controlling the sensing unit 110. In this case, the reception unit is realized by a communication device for communicating with the server 200 via a network.

### 1-3. Functional Configuration of Server

FIG. 3 shows a schematic functional configuration of the server 200 according to the first embodiment of the present disclosure. When referring to FIG. 3, the server 200 can include a reception unit 210, a storage 220, a context information acquisition unit 230, a content extraction unit 240, an output control unit 250, and a transmission unit 260. Note that the context information acquisition unit 230, the content extraction unit 240, and the output control unit 250 are realized by software with the use of, for example, a central processing unit (CPU). Note that a part or all of functions of the server 200 may be realized by the detection device 100 or the terminal device 300.

The reception unit 210 is realized by a communication device for communicating with the detection device 100 or the like via a network. For example, the reception unit 210 communicates with the detection device 100 and receives sensing data transmitted from the detection device 100. Furthermore, the reception unit 210 outputs the received sensing data to the context information acquisition unit 230. Further, the reception unit 210 can also communicate with another device via a network and receive another piece of information used by the context information acquisition unit 230 and the content extraction unit 240 described below, such as profile information of the user (hereinafter, also referred to as “user profile”) and information on content stored on another device. Note that details of the user profile will be described below.

The context information acquisition unit 230 analyzes the sensing data received by the reception unit 210 and generates context information on a state of the user. Furthermore, the context information acquisition unit 230 outputs the generated context information to the content extraction unit 240 or the storage 220. Note that details of analysis and generation of the context information in the context information acquisition unit 230 will be described below. Further, the context information acquisition unit 230 can also acquire the user profile received by the reception unit 210.

Based on the above context information, the content extraction unit 240 extracts one or more pieces of content from a content group usable by the terminal device 300 (which can include, for example, content stored on the storage 220 of the server 200, content stored on another server accessible via a network, and/or local content stored on the terminal device 300). Furthermore, the content extraction unit 240 can also output content information that is information on the extracted content to the output control unit 250 or the storage 220.

The output control unit 250 controls output of the extracted content to the user. Specifically, the output control unit 250 selects an output method, such as an output form at the time of outputting the content information to the user, the terminal device 300 to which the content information is output, and an output timing, on the basis of the content information and context information corresponding thereto. Note that details of the selection of the output method performed by the output control unit 250 will be described below. Furthermore, the output control unit 250 outputs the content information to the transmission unit 260 or the storage 220 on the basis of the selected output method.

The transmission unit 260 is realized by a communication device for communicating with the terminal device 300 or the like via a network. The transmission unit 260 communicates with the terminal device 300 selected by the output control unit 250 and transmits the content information to the terminal device 300.

### 1-4. Functional Configuration of Terminal Device

The terminal device 300 includes a mobile phone (including a smartphone), a tablet-type, notebook-type, or desktop-type PC or a TV, a portable or stationary media player (including a music player, a video display, and the like), a portable or stationary game console, a wearable computer, or the like and is not particularly limited. The terminal device 300 receives content information transmitted from the server 200 and outputs the content information to the user. Note that a function of the terminal device 300 may be realized by, for example, the same device as the detection device 100. Further, in a case where the system 10 includes a plurality of the detection devices 100, a part thereof may realize the function of the terminal device 300.

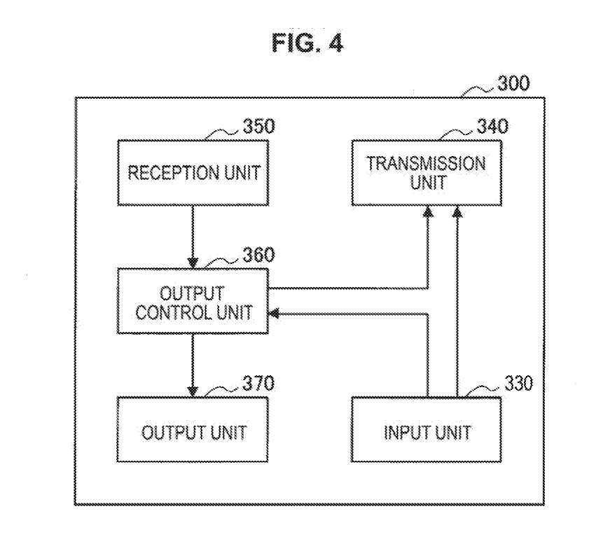

FIG. 4 shows a schematic functional configuration of the terminal device 300 according to the first embodiment of the present disclosure. As shown in FIG. 4, the terminal device 300 can include a reception unit 350, an output control unit 360, and an output unit 370.

The reception unit 350 is realized by a communication device for communicating with the server 200 via a network and receives content information transmitted from the server 200. Furthermore, the reception unit 350 outputs the content information to the output control unit 360.

The output control unit 360 is realized by software with the use of, for example, a CPU and controls output in the output unit 370 on the basis of the above content information.

The output unit 370 is configured as a device capable of outputting acquired content information to the user. Specifically, the output unit 370 can include, for example, a display device such as a liquid crystal display (LCD) or an organic electro luminescence (EL) display and an audio output device such as a speaker or headphones.

Furthermore, the terminal device 300 may further include an input unit 330 for accepting input of the user and a transmission unit 340 for transmitting information or the like from the terminal device 300 to the server 200 or the like. Specifically, for example, the terminal device 300 may change output in the output unit 370 on the basis of input accepted by the above input unit 330. In this case, the transmission unit 340 may transmit a signal for requiring the server 200 to transmit new information on the basis of the input accepted by the input unit 330.

Hereinabove, the schematic functional configurations of the system and each device according to the present embodiment have been described. Note that a configuration of a system in another embodiment is not limited to the above example, and various modifications can be made. For example, as described above, a part or all of the functions of the server 200 may be realized by the detection device 100 or the terminal device 300. Specifically, for example, in a case where the functions of the server 200 are realized by the detection device 100, the detection device 100 can include the sensing unit 110 including a sensor for providing at least one piece of sensing data and the context information acquisition unit 230 and the content extraction unit 240 (which have been described as a functional configuration of the server 200 in the above description). Further, for example, in a case where the functions of the server 200 are realized by the terminal device 300, the terminal device 300 can include the output unit 370 for outputting content, the context information acquisition unit 230, and the content extraction unit 240. Note that, in a case where all of the functions of the server 200 are realized by the detection device 100 or the terminal device 300, the system 10 does not necessarily include the server 200. Furthermore, in a case where the detection device 100 and the terminal device 300 are realized by the same device, the system 10 may be completed inside the device.

### 2. Information Processing Method

Next, an information processing method in the first embodiment of the present disclosure will be described. First, when a flow of the information processing method in the first embodiment will be roughly described, the server 200 analyzes information including sensing data regarding a state of a user detected by the detection device 100 and acquires context information indicating the state of the user obtained by the analysis. Furthermore, the server 200 extracts one or more pieces of content from a content group on the basis of the above context information.

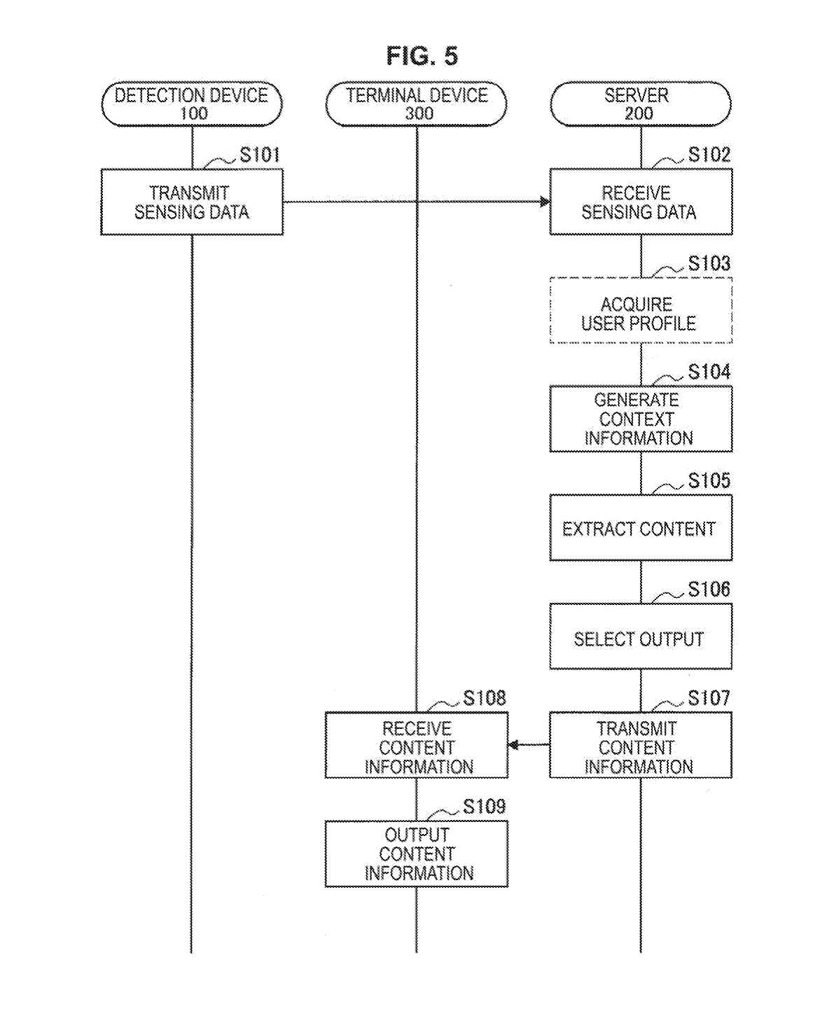

Hereinafter, details of the information processing method in the first embodiment will be described with reference to FIG. 5. FIG. 5 is a sequence diagram showing the information processing method in the first embodiment of the present disclosure.

First, in Step S101, the sensing unit 110 of the detection device 100 generates sensing data indicating a state of a user, and the transmission unit 130 transmits the sensing data to the server 200. Note that generation and transmission of the sensing data may be performed, for example, periodically or may be performed in a case where it is determined that the user is in a predetermined state on the basis of another piece of sensing data. Further, for example, in a case where the sensing unit 110 includes a plurality of kinds of sensors, generation and transmission of pieces of sensing data may be collectively implemented or may be implemented at different timings for the respective sensors.

Next, in Step S102, the reception unit 210 of the server 200 receives the sensing data transmitted from the detection device 100. The context information acquisition unit 230 acquires the received sensing data. The sensing data may be received by the reception unit 210 and then be stored on the storage 220 once and may be read out by the context information acquisition unit 230 as necessary.

Further, Step S103 may be executed as necessary, and the reception unit 210 may acquire a user profile that is information on the user via a network. The user profile can include, for example, information on the user's taste (interest graph), information on friendships of the user (social graph), and information such as a schedule of the user, image data including a face of the user, and feature data of voice of the user. Furthermore, as necessary, the context information acquisition unit 230 can also acquire, for example, various kinds of information other than the user profile, such as traffic information and a broadcast program table, via the Internet. Note that the processing order of Step S102 and Step S103 is not limited thereto, and Step S102 and Step S103 may be simultaneously performed or may be performed in the opposite order.

In Step S104, the context information acquisition unit 230 analyzes the sensing data, generates context information indicating the state of the user, and outputs the generated context information to the content extraction unit 240. Specifically, for example, the context information acquisition unit 230 may generate context information including a keyword corresponding to the acquired sensing data (in a case of sensing data regarding movement, a keyword expressing the movement; in a case of sensing data regarding voice of the user, a keyword expressing emotion of the user corresponding to the voice; in a case of sensing data regarding biological information of the user, a keyword expressing emotion of the user corresponding to the biological information; and the like). Further, the context information acquisition unit 230 may generate context information including index values in which emotions of the user obtained by analyzing the sensing data are expressed by a plurality of axes such as an axis including excitement and calmness and an axis including joy and sadness. Furthermore, the context information acquisition unit 230 may generate individual emotions as different index values (for example, excitement 80, calmness 20, and joy 60) and may generate context information including an index value obtained by integrating those index values.

Furthermore, in Step S104, in a case where position information of the user is included in the acquired sensing data, the context information acquisition unit 230 may generate context information including specific position information of the user. Further, in a case where information on a person or the terminal device 300 positioning around the user is included in the acquired sensing data, the context information acquisition unit 230 may generate context information including specific information on the person or the terminal device 300 around the user.

Herein, the context information acquisition unit 230 may associate the generated context information with a time stamp based on a time stamp of the sensing data or may associate the generated context information with a time stamp corresponding to a time at which the context information has been generated.

Further, in Step S104, the context information acquisition unit 230 may refer to the user profile at the time of analyzing the sensing data. For example, the context information acquisition unit 230 may collate the position information included in the sensing data with a schedule included in the user profile and specify a specific place where the user positions. In addition, the context information acquisition unit 230 can refer to feature data of voice of the user included in the user profile and analyze audio information included in the sensing data. Furthermore, for example, the context information acquisition unit 230 may generate context information including a keyword obtained by analyzing the acquired user profile (a keyword corresponding to the user's taste, a name of a friend of the user, or the like). In addition, the context information acquisition unit 230 may generate context information including an index value indicating a depth of a friendship of the user or action schedule information of the user.

Next, in Step S105, the content extraction unit 240 extracts one or more pieces of content from pieces of content that can be acquired via a network on the basis of the context information generated by the context information acquisition unit 230. Then, the content extraction unit 240 outputs content information that is information on the extracted content to the output control unit 250 or the storage 220.

Specifically, the content extraction unit 240 extracts, for example, content having the content suitable for the state of the user expressed by the keyword or the like included in the context information. At this time, the content extraction unit 240 can also extract content having a format (text file, still image file, moving image file, audio file, or the like) based on the position information of the user included in the context information or the terminal device 300 used by the user. Furthermore, the content extraction unit 240 may calculate a matching degree indicating a degree of matchability between each extracted piece of content and context information used at the time of extraction and output the calculated matching degree as content information of each piece of content.

Next, in Step S106, the output control unit 250 selects an output method at the time of outputting the content information to the user, the terminal device 300 to which the content information is output, an output timing, and the like and outputs information on the selection to the transmission unit 260 or the storage 220. The output control unit 250 performs the above selection on the basis of the above content information and the context information relating thereto.

Specifically, the output control unit 250 selects an output method of content, for example, whether or not a substance such as video or audio of the extracted content is output, whether or not a list in which titles of pieces of content and the like are arranged is output, or whether or not content having the highest matching degree is recommended by an agent. For example, in a case where the output control unit 250 outputs a list in which titles of pieces of content and the like are arranged, pieces of information on individual pieces of content may be arranged in order based on the calculated matching degrees or may be arranged on the basis of, for example, reproduction times instead of the matching degrees. Further, the output control unit 250 selects one or more devices from the terminal devices 300 as an output terminal for outputting content information. For example, the output control unit 250 specifies the terminal device 300 positioning around the user on the basis of the context information and selects a piece of content having a format or size that can be output by the terminal device 300 from the extracted pieces of content. Furthermore, for example, the output control unit 250 selects a timing at which the content information is output on the basis of the action schedule information of the user included in the context information or determines a sound volume and the like at the time of reproducing the content in accordance with a surrounding environment around the user on the basis of the position information of the user.

In Step S107, the transmission unit 260 communicates with the terminal device 300 via a network and transmits the content information on the basis of the selection by the output control unit 250.

Next, in Step S108, the reception unit 350 of the terminal device 300 receives the above content information. Then, the output control unit 360 controls the output unit 370 on the basis of the received content information.

In Step S109, the output unit 370 is controlled by the output control unit 360 and outputs the content information (for example, information such as content substance or title) to the user.

Further, although not shown in the sequence of FIG. 5, for example, the server 200 can acquire information on content viewed by the user as a viewing history of the user after Step S109. In this case, the server 200 can include a history acquisition unit (not shown in FIG. 3), and the history acquisition unit can acquire information on the user's taste by learning the acquired viewing history. Furthermore, it is possible to use this acquired information on the user's taste for the next content extraction. In addition, the server 200 can acquire evaluation of the extracted content from the user. In this case, the input unit 330 included in the terminal device 300 accepts input from the user, and the above evaluation is transmitted from the terminal device 300 to the server 200. In this case, the server 200 further includes an evaluation acquisition unit (not shown in FIG. 3), and the evaluation acquisition unit accumulates and learns the above evaluation, thereby acquiring information on the user's taste.

As a further modification example, the server 200 may accept input of a keyword for extraction from the user. A timing of acceptance may be a timing before extraction of content or may be a timing after content information of content extracted once is output to the user. Further, a device that accepts input can be an input unit of the server 200, the sensing unit 110 of the detection device 100, or the like and is not particularly limited.

Hereinafter, an example of information processing according to the first embodiment of the present disclosure will be described by using specific examples. Note that the following examples are merely examples of the information processing according to the first embodiment, and the information processing according to the first embodiment is not limited to the following examples.

### 2-1. First Example

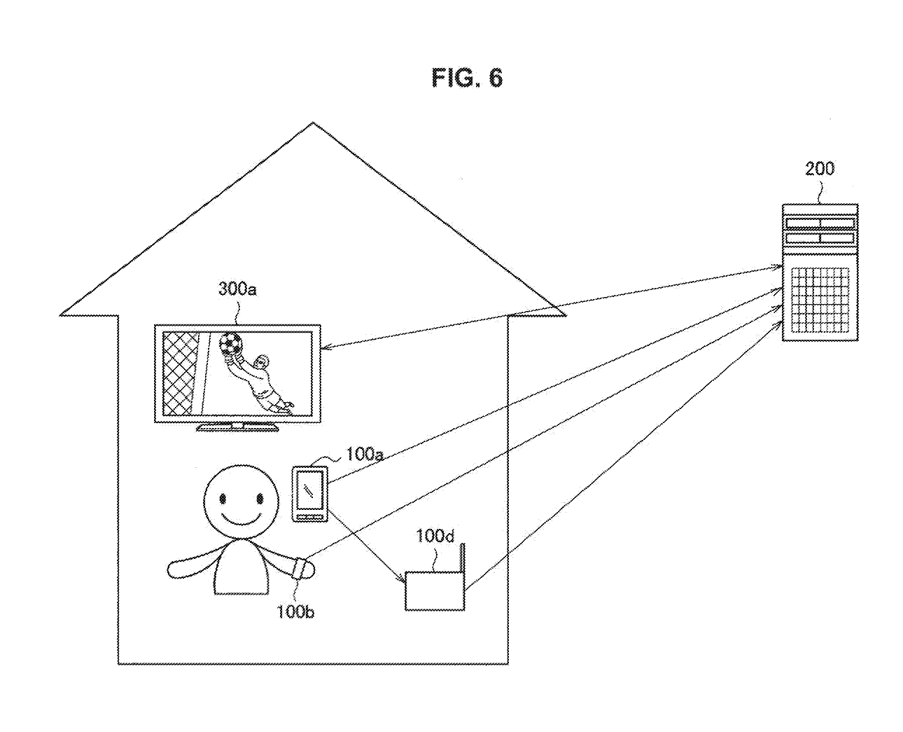

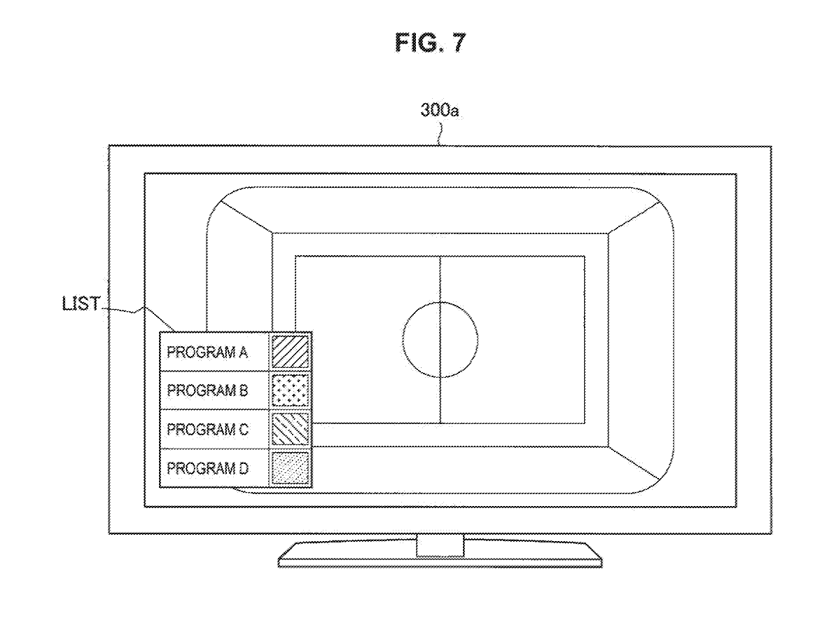

Hereinafter, a first example will be described more specifically with reference to FIG. 6 and FIG. 7. FIG. 6 and FIG. 7 are explanatory views for describing the first example. In the first example, as shown in FIG. 6, there is assumed a case where a user watches a relay broadcast of soccer game on TV in a living room of the user's home.

In the present example, a smartphone 100a carried by the user and a wristwear 100b function as the detection device 100. The smartphone 100a detects position information indicating that the user is in the living room of the user's home on the basis of, for example, an access point 100d and radio field intensity of Wi-Fi via which the smartphone 100a can communicate and transmits sensing data based on the detection to the server 200. Furthermore, the server 200 can separately access a TV 300a specified to exist in the living room of the user's home on the basis of information registered by the user via the Internet and acquire information on a state of the TV 300a (information such as a state of a power supply and a received channel) on the basis of the above sensing data. Based on the above information, the context information acquisition unit 230 of the server 200 can grasp a state in which the user is in the living room of the user's home, the TV 300a exists as the terminal device 300 positioning around the user, and the above TV 300a is turned on and receives a channel 8.

Next, the context information acquisition unit 230 acquires a program table of the channel 8 that can be used on a network via the reception unit 210. In an example shown in FIG. 6, based on the acquired information, the context information acquisition unit 230 can specify that a program estimated to be currently viewed by the user is a relay broadcast of a soccer game and can specify names of soccer teams playing the game, starting date and time of the game, and the like.

Herein, there is assumed a case where the user performs movement of raising his/her arm in the middle of the above context (the user currently views a soccer relay broadcast on TV in the living room). At this time, an acceleration sensor included in the wristwear 100b transmits sensing data indicating a change in acceleration generated by raising his/her arm to the server 200. In the server 200, the context information acquisition unit 230 specifies that the user's movement “raising his/her arm” has occurred by analyzing the transmitted sensing data. The movement “raising his/her arm” has occurred in the context “currently viewing the soccer relay broadcast” that had already been specified, and therefore the context information acquisition unit 230 generates context information indicating that “the user got excited and raised his/her arm while viewing the soccer relay broadcast”.

Next, in the server 200, the content extraction unit 240 extracts, for example, content “an exciting scene of a game of soccer” on the basis of the generated context information. At this time, the content extraction unit 240 may extract content by using a keyword “soccer”, “excitement”, or the like included in the context information or may extract content by using, for example, a feature vector indicating the kind of sport or a feature of a scene. Furthermore, the content extraction unit 240 can grasp a state in which the user currently views the soccer relay broadcast on the TV 300a in the living room on the basis of the context information and therefore limits content to be extracted to a moving image having a size suitably output by the TV 300a and extracts the content.

In the example shown in FIG. 7, a plurality of pieces of content are extracted by the content extraction unit 240 as content suitable for the state of the user indicated by the context information. In this case, a matching degree indicating a degree of matchability between each extracted piece of content and the context information used at the time of extraction is calculated, and the calculated matching degree is included in content information on each piece of content. Furthermore, the output control unit 250 selects to output the content information in a list form on the TV 300a. Specifically, the output control unit 250 of the server 200 selects to output a list in which titles and thumbnails of the individual pieces of content are arranged in order based on the matching degrees of the individual pieces of content (for example, content information of a piece of content having a high matching degree is shown at the top). In addition, the output control unit 250 refers to information on the soccer relay broadcast and selects to output the list at a timing at which a first half of the game is completed and half-time starts.

By the processing described above in the server 200, as shown in FIG. 7, the list (LIST) in which the titles and the thumbnails of the extracted pieces of content are arranged is displayed on a screen of the TV 300a when the half-time starts. Furthermore, when the user selects content that the user desires to view from the above list, the selected content is reproduced. In this case, selection of the content by the user is input by using a remote controller of the TV 300a (example of the input unit 330 of the terminal device 300) or the like.

In the first example described above, the wristwear 100b can detect movement of the user that cannot be easily expressed by words, such as movement of raising his/her arm, and the server 200 can extract content based on the movement. At this time, a state in which the user currently watches the soccer relay broadcast on the TV 300a in the living room is also grasped on the basis of the position information provided by the smartphone 100a and the information provided from the TV 300a, and therefore it is possible to extract more appropriate content.

Further, in the present example, content is extracted by using, as a trigger, detection of movement executed by the user without intending extraction of content. With this, it is possible to extract content in which a potential desire of the user (desire to watch other exciting scenes of soccer relay broadcasts) is reflected, and therefore the user can enjoy the content with unexpectedness or surprise. Furthermore, in the present example, the terminal device 300 (TV 300a) on which the user views the extracted content and a state of output in the terminal device 300 (the soccer relay broadcast is currently output, and the game is soon stopped and half-time starts) are automatically specified, and therefore it is possible to output the extracted content to an optimal terminal device at an optimal timing to the user. Therefore, the user can enjoy the extracted content more comfortably.

Furthermore, for example, in a case where the user sees the list and desires to extract a moving image of a certain player from the pieces of content appearing in the list, the user may input a keyword for content extraction (for example, a name of the player). In this case, the user can input the above keyword by operating the smartphone 100a carried by the user. That is, in this case, the smartphone 100a functions as the detection device 100 for providing position information of the user and functions also as the terminal device 300 for accepting operation input of the user. In the server 200 that has received the input keyword, the content extraction unit 240 further extracts one or more pieces of content matching the keyword from the plurality of pieces of content that have already been extracted. As described above, the server 200 can perform extraction by using not only the context information obtained by analyzing the sensing data but also the keyword, and therefore it is possible to extract content more appropriate for the user.

In the above case, in a case where the keyword input from the user has various meanings, the context information acquisition unit 230 can specify a meaning intended by the user by analyzing the context information obtained from the sensing data together with the keyword. Specifically, in a case where a keyword “omoshiroi” is input from the user, the keyword “omoshiroi” has meanings “funny”, “interesting”, and the like. When the keyword is input, the context information acquisition unit 230 analyzes, for example, a brain wave of the user detected by the biosensor worn on a head of the user and grasps a context of the user indicating that “the user is concentrating”. In this case, the server 200 specifies that a meaning of the keyword “omoshiroi” intended by the user is “interesting” on the basis of the context information indicating that “the user is concentrating” and extracts content based on the keyword “interesting”.

### 2-2. Second Example

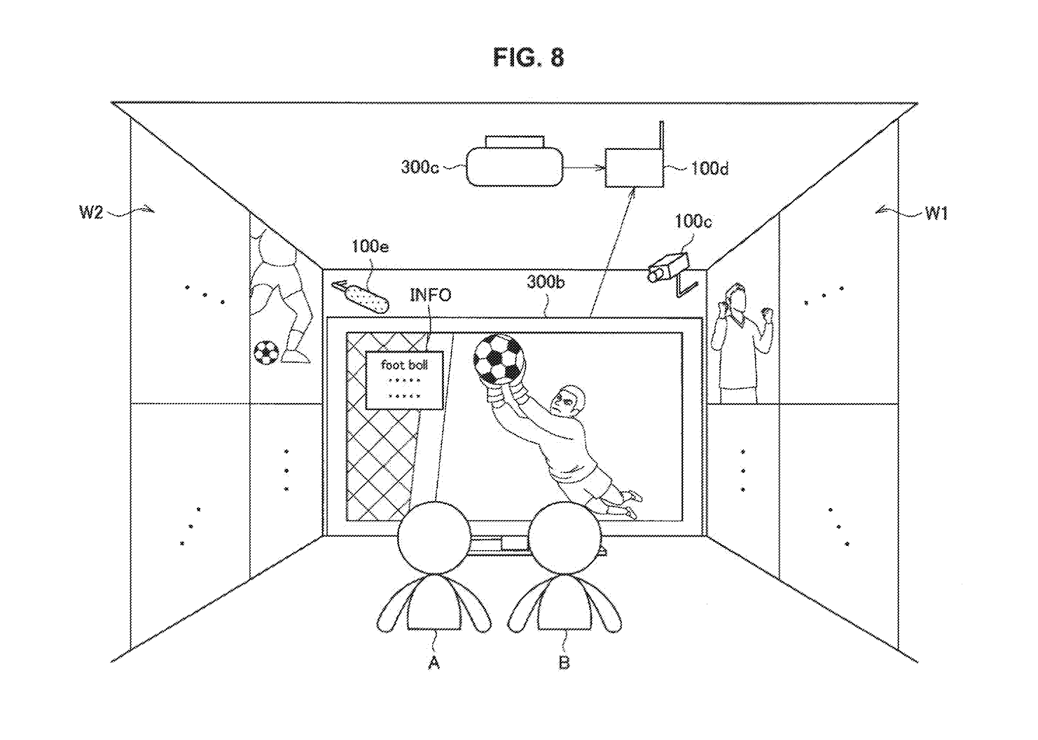

Hereinafter, a second example will be described more specifically with reference to FIG. 8. FIG. 8 is an explanatory view for describing the second example. In the second example, as shown in FIG. 8, there is assumed a case where a user A is in a living room of the user A's home and has a pleasant talk with a user B who is a friend of the user A while watching a soccer relay broadcast on TV.

Faces of the users A and B are imaged by an imaging device 100c placed in the living room of the user A's home, the imaging device 100c corresponding to the detection device 100. The imaging device 100c transmits sensing data including position information of the imaging device 100c and face images of the users A and B to the server 200. In the server 200, the context information acquisition unit 230 refers to face image data included in a user profile acquired via a network and specifies that the face images included in the transmitted sensing data are face images of the users A and B. Then, the context information acquisition unit 230 grasps that the users A and B are in the living room of the user A's home on the basis of the above information included in the sensing data. Furthermore, the context information acquisition unit 230 also grasps that the user A and the user B currently have a pleasant talk on the basis of a moving image of movement of the users A and B (for example, the users A and B face each other sometimes) transmitted from the imaging device 100c.

In the server 200, the context information acquisition unit 230 acquires user profiles including interest graphs of the respective users A and B via a network. Then, the context information acquisition unit 230 can grasp tastes of the respective users A and B (for example, “The user A has a good time when the user A watches a variety program.”, “A favorite group of the user A is “ABC37”.”, and “The way the user B spends a fun time is playing soccer.”) on the basis of the acquired interest graphs.

Meanwhile, the Wi-Fi access point 100d placed in the living room of the user A's home communicates with a TV 300b placed in the living room of the user A's home and a projector 300c for projecting video onto a wall surface of the living room. When the Wi-Fi access point 100d transmits information on this communication to the server 200, the context information acquisition unit 230 of the server 200 can specify that there are the TV 300b and the projector 300c as the usable terminal device 300.

Herein, there is assumed a case where, in the middle of the above context (the users A and B currently have a pleasant talk), the user A enjoys talking and gives a laugh. A microphone 100e placed in the living room of the user A's home together with the imaging device 100c detects the above laugh and transmits sensing data including audio data of the laugh to the server 200. In the server 200, the context information acquisition unit 230 refers to feature information of voice included in the above acquired user profile and specifies that a laugh of the user A is included in the transmitted sensing data. Furthermore, the context information acquisition unit 230 that has specified a person who gave the laugh refers to information on a correlation between voice of the user A included in the above user profile and emotion (an enjoyable feeling in a case of a loud laugh, a sad feeling in a case of sobbing voice, and the like) and generates context information including a keyword (for example, “enjoyable”) indicating emotion of the user A at the time of giving the laugh. Note that, in the second example, description has been made assuming that the laugh of the user A is detected by the microphone 100e. However, for example, sound detected by the microphone 100e may be a shout for joy such as “Wow!”, sniffing sound, coughing sound, or an uttered voice. Further, the microphone 100e may detect sound caused by movement of the user B.

In the present example, the content extraction unit 240 of the server 200 can extract content by two methods. In a first method, the content extraction unit 240 extracts, for example, content of a variety program in which “ABC37” appears on the basis of the keyword “enjoyable” included in the context information and the user A's taste (“The user A has a good time when the user A watches a variety program.” and “A favorite group of the user A is “ABC37”.”).

Meanwhile, in a second method, the content extraction unit 240 extracts content by using not only a plurality of kinds of information used in the first method but also the user B's taste (The user B spends a fun time playing soccer.) included in the context information. In this case, content to be extracted is, for example, content of a variety program regarding soccer such as a variety program in which a soccer player and “ABC37” appear or a variety program in which “ABC37” challenges soccer.

In the present example, the content extraction unit 240 may extract content by using any one of the above first and second methods or may extract content by both the methods.

Herein, the server 200 communicates with the TV 300b via the Wi-Fi access point 100d and therefore recognizes that the TV 300b has been turned on. Meanwhile, the server 200 also recognizes that the projector 300c has not been turned on by similar communication. In this case, the context information acquisition unit 230 generates context information further including information indicating that the users A and B currently view the TV 300b. The output control unit 250 selects the projector 300c as the terminal device 300 to which content information is output so as not to interrupt viewing of the TV 300b on the basis of the above context information. Furthermore, the output control unit 250 selects the projector 300c to project a list including titles of individual moving images and still images of representative scenes of the individual moving images from the content information.

Further, in the example shown in FIG. 8, a plurality of pieces of content are extracted by each of the two methods, and therefore the output control unit 250 selects to separately output content information extracted by each method. Specifically, as shown in FIG. 8, the projector 300c can project videos onto two wall surfaces W1 and W2, respectively, in the vicinity of the TV 300b in the living room of the user's home. In view of this, the output control unit 250 determines that pieces of content information of variety programs extracted by the first method are projected onto the right wall surface W1 and pieces of content information of variety programs regarding soccer extracted by the second method are projected onto the left wall surface W2.

Furthermore, in the example shown in FIG. 8, the output control unit 250 refers to information such as first broadcast date and time associated with each extracted content and arranges newer pieces of content on parts of the wall surfaces W1 and W2 closer to the TV 300b. For example, the newest pieces of content information are projected onto parts of the wall surfaces W1 and W2 closest to the TV. On the contrary, the oldest pieces of content information are projected onto parts of the wall surfaces W1 and W2 farthest from the TV. Furthermore, in a case where there is content especially recommended on the basis of the context information and the like (for example, the newest content is especially recommended), as shown in FIG. 8, a small display of content information (INFO) of the recommended content may be performed also on an upper left part of the screen of the TV 300b.

Furthermore, when the user A selects content that the user desires to view from the projected content information, the selected content is reproduced on the screen of the TV 300b. At this time, the user A may select content by using, for example, a controller capable of selecting a position in the images projected onto the wall surfaces W1 and W2 or may select content by voice input, e.g., reading a title of content or the like. In a case of voice input, uttered voice of the user A may be detected by the microphone 100e.

In the second example described above, even in a case of a state of the user that cannot be easily expressed by words, such as emotion of the user A, it is possible to extract content based on the state of the user. Further, the context information acquisition unit 230 refers to the user profile including information on a relationship between movement of the user and emotion at the time of analyzing the sensing data, and therefore it is possible to perform analysis more accurately. Furthermore, the context information acquisition unit 230 extracts content also on the basis of information on the user B's taste included in the user profile, and therefore it is possible to extract content that the users A and B can simultaneously enjoy.

### 2-3. Third Example

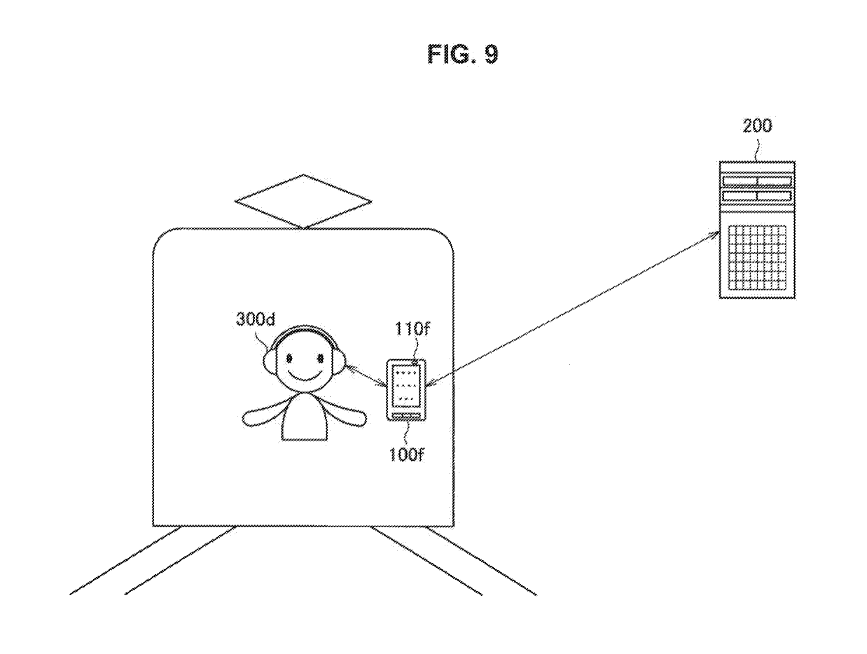

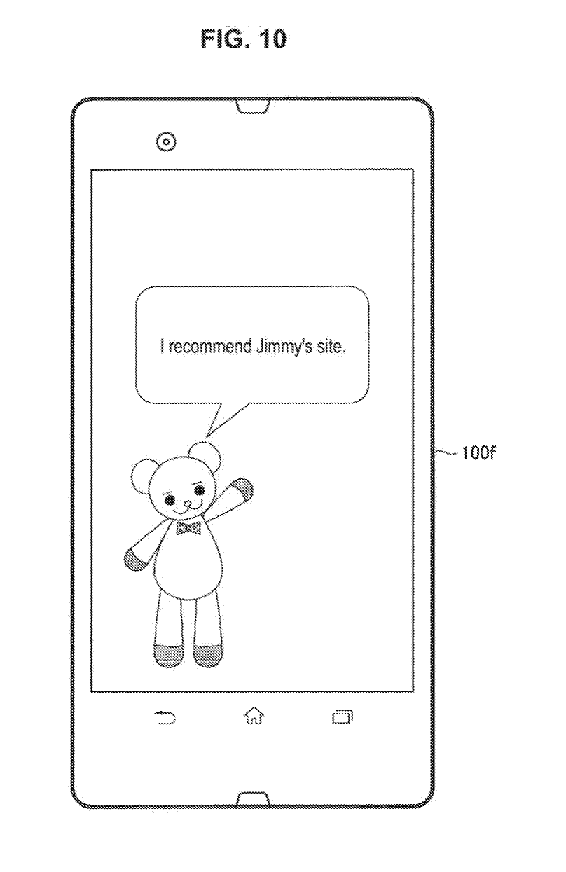

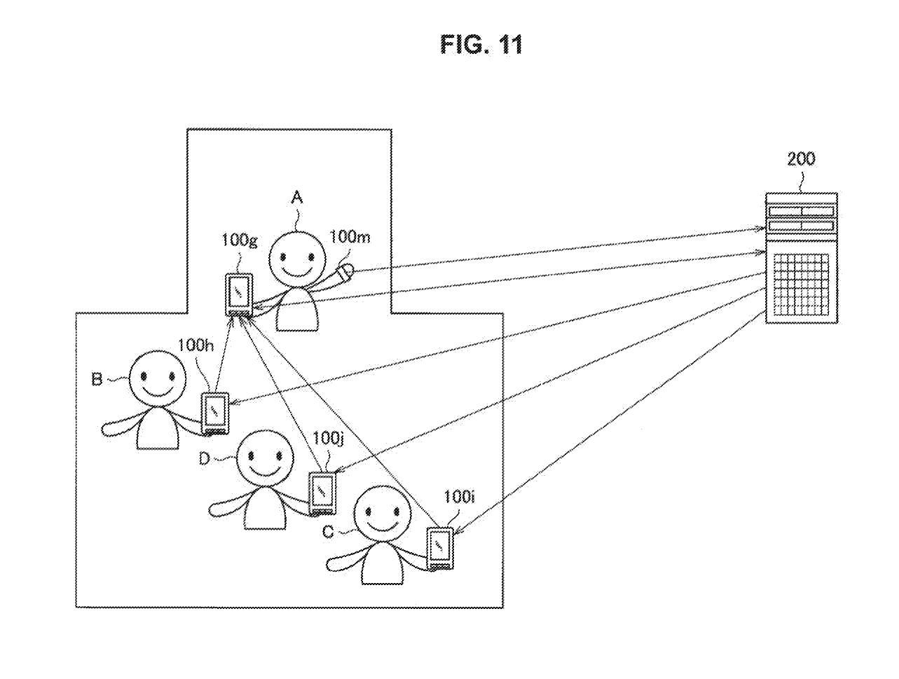

Hereinafter, a third example will be described more specifically with reference to FIG. 9 and FIG. 10. FIG. 9 and FIG. 10 are explanatory views for describing the third example. In the third example, as shown in FIG. 9, there is assumed a case where a user rides on a train and watches a screen of a smartphone 100f while listening to music.

The user carries the smartphone 100f serving as the detection device 100, and the smartphone 100f detects position information of the user by using a GNSS receiver included in the smartphone 100f and transmits sensing data based on the above detection to the server 200. Furthermore, the smartphone 100f communicates with headphones 300d worn on the user via Bluetooth (registered trademark) and transmits audio signals for outputting music to the headphones 300d. The smartphone 100f transmits information indicating that the user uses the headphones 300d together with the above position information to the server 200.

Meanwhile, in the server 200, the context information acquisition unit 230 acquires not only the information transmitted from the smartphone 100f as described above but also a user profile including schedule information via the reception unit 210 through a network. Then, the context information acquisition unit 230 grasps that the user is in a train on the basis of the position information of the user received from the smartphone 100f and the schedule information of the user (more specifically, the user is on the way to work and is riding on a subway train on Line No. 3). Furthermore, the context information acquisition unit 230 also grasps a state in which the user uses the headphones 300d together with the smartphone 100f by analyzing information included in the sensing data.

Next, there is assumed a case where the user reads a blog of a friend on a screen of social media displayed on the smartphone 100f and has a happy expression on his/her face. A camera 110f included in the smartphone 100f captures an image of the above expression of the user. The captured image is transmitted to the server 200. In the server 200, the context information acquisition unit 230 analyzes the image and specifies that the expression of the user is “happy expression”. Furthermore, the context information acquisition unit 230 generates context information including a keyword (for example, “happy”) corresponding to emotion of the user expressed by such expression. Note that the above keyword is not limited to a keyword that expresses emotion of the user having an expression on his/her face and may be, for example, a keyword such as “cheering up” in a case of a sad expression.

The content extraction unit 240 extracts content that can be output by the smartphone 100f on the basis of the keyword “happy” included in the context information. Furthermore, at the time of the above extraction, the content extraction unit 240 may recognize that the user has ten minutes left until the user gets off the train on the basis of the schedule information included in the user profile and, in a case of a moving image or audio, may extract only content having a reproduction time of ten or less minutes. As a result, the content extraction unit 240 extracts a blog of the user in which a happy event is recorded, a news site in which a happy article is written, and music data of a musical piece with which the user feels happy. The server 200 outputs content information (title, format, and the like) on the extracted content.

In the server 200, the output control unit 250 refers to information of the usable terminal device 300 included in the context information and selects the smartphone 100f as the terminal device 300 for outputting content information. In other words, in the present example, the smartphone 100f functions as the detection device 100 and also as the terminal device 300. The content information transmitted from the server 200 is displayed on the screen of the smartphone 100f. In this case, as shown in, for example, FIG. 10, an agent is displayed on the screen of the smartphone 100f, and a display in which the agent recommends the extracted content (for example, a character is displayed on the screen and “I recommend Jimmy's site!” is displayed in a balloon of the character) is performed. In this case, when the user operates the smartphone 100f, it is possible to reproduce desired content. Further, by operating the smartphone 100f, the user may input evaluation of the reproduced content and, furthermore, may input not only evaluation of the content but also evaluation of a method of outputting the content (output timing and the like).

Note that, in the above example, in a case where there is no time until the user gets off the train, only music data may be extracted and output so as not to interrupt transfer of the user. In this case, the music data is output from the headphones 300d via the smartphone 100f. Further, for example, in a case where the user currently drives an automobile, only content that can be reproduced by a speaker placed in the automobile may be extracted.

According to the third example, the server 200 can extract and output content in accordance with action schedule information of the user obtained by analyzing the user profile. Therefore, extraction and output of content is performed more suitably in accordance with a state of the user, and thus the user can enjoy the content more comfortably.

### 2-4. Fourth Example

Hereinafter, a fourth example will be described more specifically with reference to FIG. 11. FIG. 11 is an explanatory view for describing the fourth example. In the fourth example, as shown in FIG. 11, there is assumed a case where a user A spends break time with friends (friends B, C, and D) in a classroom at a school.

As in the first example, the user A carries a smartphone 100g serving as the detection device 100, and position information of the user A is detected by the smartphone 100g. Furthermore, the smartphone 100g communicates with smartphones 100h, 100i, and 100j carried by the friends B, C, and D around the user A via Bluetooth (registered trademark) and therefore detects the smartphones 100h, 100i, and 100j as terminal devices positioning therearound. The smartphone 100g transmits information indicating the detected other terminal devices (in other words, the smartphones 100h, 100i, and 100j) to the server 200. Further, the smartphone 100g transmits the position information of the user A acquired by a GNSS receiver, a Wi-Fi communication device, or the like to the server 200.

In the server 200, the context information acquisition unit 230 grasps a state in which the user A is in the classroom at the school on the basis of the position information received from the smartphone 100g. Furthermore, the context information acquisition unit 230 recognizes the smartphones 100h, 100i, and 100j as other terminal devices positioning around the user A on the basis of the information received from the smartphone 100g. In addition, the server 200 may refer to account information associated with each of the above smartphones via a network and specify the friends B, C, and D who are possessors of the smartphones 100h, 100i, and 100j as persons around the user A. Furthermore, in the server 200, the context information acquisition unit 230 acquires not only the information transmitted from the smartphone 100g as described above but also a user profile including schedule information of the user A via the reception unit 210 through a network. The context information acquisition unit 230 can also grasp context in which the user A is at break time on the basis of the above schedule information.

Furthermore, the context information acquisition unit 230 may extract information on the friends B, C, and D specified as persons around the user A from a social graph included in the user profile of the user A. More specifically, the context information acquisition unit 230 generates context information including information on friendships between the user A and the friends B, C, and D (index value of a degree of intimacy or a relationship, for example, 5 in a case of a best friend or family member, 4 in a case of a classmate, and 1 in a case of a neighbor) on the basis of the acquired social graph.

The content extraction unit 240 may extract content by reflecting the friendships between the user A and the friends B, C, and D on the basis of the context information including such information. Specifically, for example, in a case where it is recognized that the friends B, C, and D do not have an especially close relationship with the user A on the basis of friendship information, the content extraction unit 240 does not extract private content of the user A (for example, a moving image of the user A captured by a home video camera). Note that, in a case where the friends B, C, and D have an especially close relationship with the user A, the content extraction unit 240 may extract private content of the user A specified to be openable in advance. Further, disclosure level information in which a disclosure level at which content can be disclosed is written for each person (information in which a disclosure range of content is set for each person, for example, private content is opened to a friend E and private content is not open to a friend F) may be prepared by the user A in advance, and content may be extracted in accordance with this disclosure level information.

Next, there is assumed a case where the user A performs movement of taking a shot in tennis at break time. As in the first example, an acceleration sensor included in a wristwear 100m worn on an arm of the user A transmits sensing data indicating an acceleration change generated due to the above movement to the server 200. In the server 200, the context information acquisition unit 230 specifies that the user A has performed the movement of taking a shot in tennis by analyzing the transmitted sensing data. Furthermore, the context information acquisition unit 230 generates context information including keywords (for example, “tennis” and “shot”) corresponding to the above movement of the user A.

In the server 200, the context extraction unit 240 extracts a moving image of a shot in tennis on the basis of the keywords “tennis” and “shot” included in the context information and terminal device information and outputs content information on the extracted moving image. At the time of extraction, private content of the user A is not extracted as described above, and therefore, for example, a moving image in which the user A plays tennis, which is captured by a home video camera, is not extracted. Note that, in the present example, a single moving image is assumed to be extracted.

In the server 200, the output control unit 250 refers to the terminal device information included in the context information and selects the smartphones 100g, 100h, 100i, and 100j as the terminal device 300 for outputting content information. More specifically, the number of extracted moving images is one, and therefore the output control unit 250 selects to display this moving image on the smartphone 100g carried by the user A and simultaneously display the moving image also on the smartphones 100h, 100i, and 100j.

Furthermore, in a case where the friend B shouts “Great!” when the friend B watches content displayed on the smartphone 100h, the shout of the friend B is detected by a microphone included in the smartphone 100h, and sensing data based on this detection is transmitted to the server 200. In this case, the server 200 performs generation of context information and extraction processing of content by using acquisition of the above sensing data as a trigger, and the extracted content is output to the user A and the friends B, C, and D. In a case where a new state of the user A or the like is further detected, the server 200 extracts new content based on the detected new state of the user A or the like.

Note that, in the above example, content information is simultaneously output to smartphones. However, the present disclosure is not limited thereto, and the content information may be displayed on the smartphones at different timings. For example, in a case where the friend C operates the smartphone 100i, the content information may be displayed on the smartphone 100i after termination of the operation is confirmed at a timing different from timings of the other smartphones. Further, a timing at which content is displayed on each smartphone and content that the user desires to view may be input by the user A operating the smartphone 100g. Furthermore, in a case where, among the friends around the user A, the friend D carries a feature phone, the content can be displayed as follows. For example, content including text and a still image corresponding to the content displayed on each smartphone may be displayed on the feature phone of the friend D in accordance with an ability of a screen display function of the feature phone.

In the fourth example, it is possible to output content information not only to the smartphone 100g carried by the user A but also to smartphones carried by friends around the user A and share content with the friends therearound. Furthermore, the server 200 extracts content in accordance with friendship information of the user A, and therefore private video or the like that the user A does not desire to show to the friends or the like is not displayed on the smartphones of the friends, and thus the user A can enjoy the content at ease.

### 3. Second Embodiment

In a second embodiment, context information indicating a state of a user is separately used as metainformation of content corresponding to the context information. This metainformation is used when, for example, extraction of content described in the first embodiment is performed. In other words, in the present embodiment, in a case where content is extracted, it is possible to use metainformation associated with the content (corresponding to past content information) and context information (for example, collate or compare the metainformation with the context information). Therefore, it is possible to extract content more suitable for a state of the user.

Hereinafter, the second embodiment of the present disclosure will be described with reference to the drawings. Note that a system according to the second embodiment includes a detection device 100, a terminal device 300, and a server 400. Note that functional configurations of the detection device 100 and the terminal device 300 are similar to the functional configurations thereof in the first embodiment, and therefore description thereof is herein omitted.

### 3-1. Functional Configuration of Server

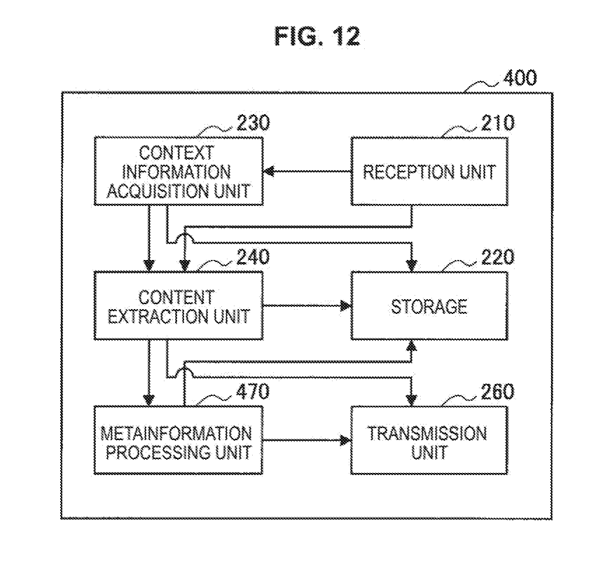

A schematic functional configuration of the server 400 according to the second embodiment will be described. FIG. 12 shows the schematic functional configuration of the server 400 according to the second embodiment. As is clear from FIG. 12, the server 400 according to the second embodiment, as well as the server 200 according to the first embodiment, can include a reception unit 210, a storage 220, a context information acquisition unit 230, a content extraction unit 240, and a transmission unit 260. Furthermore, the server 400 can also include a metainformation processing unit 470. Note that the context information acquisition unit 230, the content extraction unit 240, and the metainformation processing unit 470 are realized by software with the use of, for example, a CPU or the like.

The metainformation processing unit 470 associates context information generated by the context information acquisition unit 230 as metainformation with one or more pieces of content extracted on the basis of the above context information by the content extraction unit 240. In addition, the metainformation processing unit 470 can also output the metainformation based on the context information to the transmission unit 260 or the storage 220. Note that the reception unit 210, the storage 220, the context information acquisition unit 230, the content extraction unit 240, and the transmission unit 260 of the server 400 are similar to those units in the first embodiment, and therefore description thereof is herein omitted.

### 3-2. Information Processing Method

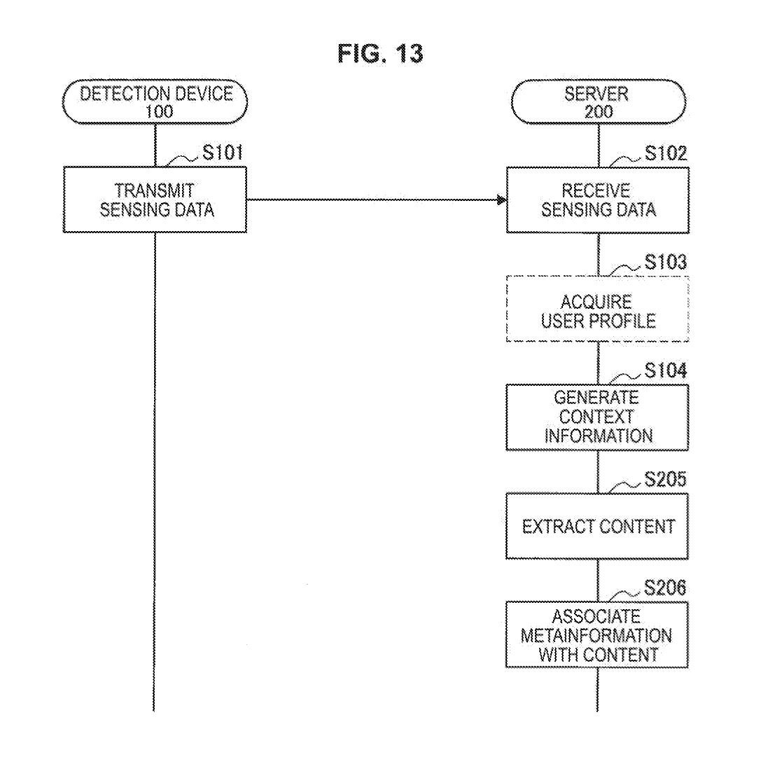

FIG. 13 is a sequence diagram showing a method of information processing in the second embodiment of the present disclosure. The method of the information processing in the second embodiment will be described with reference to FIG. 13. First, in Step S101 to Step S104 are executed. Those steps are similar to the steps shown in FIG. 5 in the first embodiment, and therefore description thereof is herein omitted.

In Step S205, based on generated context information, the content extraction unit 240 of the server 400 extracts one or more pieces of content corresponding to the context information from a large number of pieces of content that can be acquired via a network. Specifically, the content extraction unit 240 extracts content such as a moving image and a musical piece viewed/listened to by the user on the basis of position information of the user included in the context information, terminal device information used by the user, and the like. More specifically, the content extraction unit 240 may extract a moving image or the like associated with a time stamp of the same time as a time at which sensing data has been acquired. Then, the server 400 outputs content information on the extracted content to the metainformation processing unit 470 or the storage 220.

In Step S206, the metainformation processing unit 470 associates the generated context information as metainformation with the extracted content. The extracted content is associated not only with the information used in extraction in Step S205 but also with another piece of information included in the context information (for example, biological information of the user obtained by analyzing the sensing data). Then, the metainformation processing unit 470 outputs the information on the content associated with the metainformation based on the context information to the transmission unit 260 or the storage 220.

Although not shown in FIG. 13, at the time of processing similar to the processing in the first embodiment (extraction of content to be output in the terminal device 300) after Step S206, in the server 400, it is possible to use the metainformation associated with the content by the metainformation processing unit 470. Specifically, the content extraction unit 240 compares and collates the metainformation associated with the content (including information corresponding to past context information) with the context information newly acquired by the context information acquisition unit 230. With this, it is possible to extract content more suitable for a state of the user.

Hereinafter, an example of the information processing according to the second embodiment of the present disclosure will be described by using a specific example. Note that the following example is merely an example of the information processing according to the second embodiment, and the information processing according to the second embodiment is not limited to the following example.

### 3-3. Fifth Example

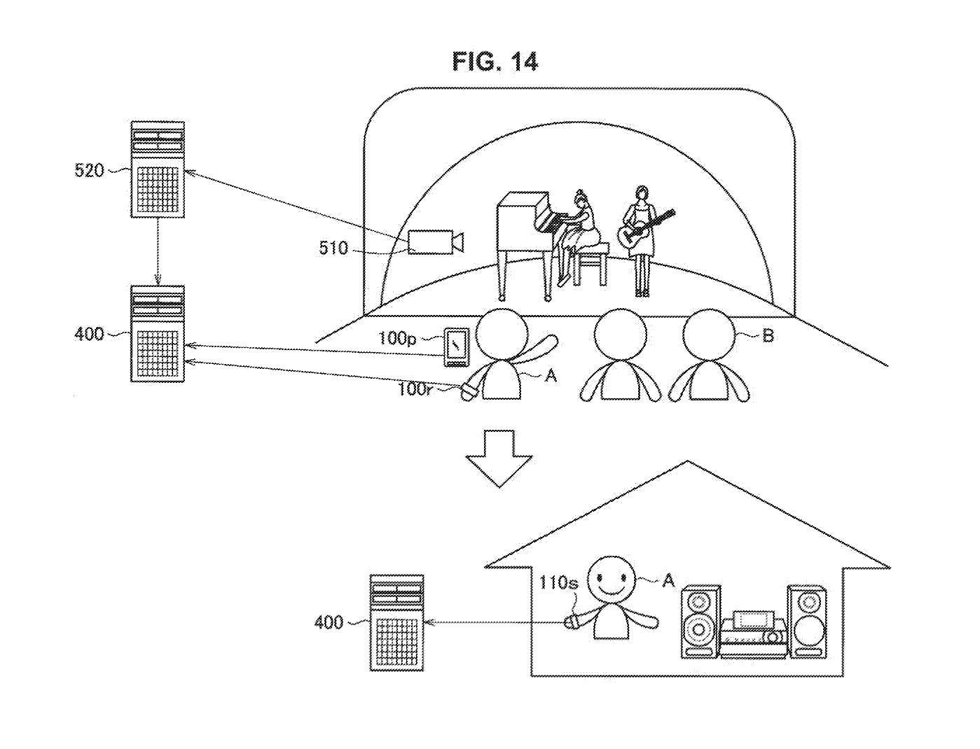

Hereinafter, a fifth example will be described more specifically with reference to FIG. 14. FIG. 14 is an explanatory view for describing the fifth example. In the fifth example, as shown in an upper part of FIG. 14, there is assumed a case where a user A appreciates music at an outdoor concert hall.

As in the first example, the user A carries a smartphone 100p as the detection device 100, and position information of the user A is detected by the smartphone 100p. Furthermore, the smartphone 100p transmits sensing data based on the above detection to the server 400. Then, in the server 400, the context information acquisition unit 230 analyzes the acquired sensing data and grasps the position information of the user A indicating that the user A is at the outdoor concert hall. Furthermore, the context information acquisition unit 230 acquires schedule information on the outdoor concert hall via a network on the basis of the above position information and specifies a concert performed at the above concert hall.

Next, there is assumed a case where the user A gets excited while the user A is appreciating the concert. A pulse sensor included in a wristwear 100r attached to a wrist of the user A as the detection device 100 detects a pulse of the user A in an excitement state and transmits sensing data to the server 400. In the server 400, the context information acquisition unit 230 analyzes the sensing data and generates context information including pulse information of the user.

Note that, in a case where sensing data based on which a fact that a friend B who is a friend of the user A appreciates the same concert at the above concert hall can be grasped is detected, information obtained by analyzing the sensing data may also be included in the context information.

Next, the content extraction unit 240 of the server 400 extracts one or more pieces of content on the basis of information on the specified concert and a time stamp of the sensing data. More specifically, the content extraction unit 240 extracts content regarding the above concert associated with a time stamp of a time same as or close to a time indicated by the above time stamp. The extracted content is, for example, a moving image of the above concert captured by a camera 510 placed at the concert hall and recorded on a content server 520, musical piece data performed at the above concert, and tweets regarding the concert posted by members of an audience of the above concert.

In the server 400, the metainformation processing unit 470 associates the context information that has already been generated as metainformation with the extracted content. Furthermore, the metainformation processing unit 470 outputs the associated metainformation.

Furthermore, there will be described an example where content is extracted with the use of the metainformation by performing processing similar to the processing in the first embodiment after the above processing in the present example is executed. In the following description, as shown in a lower part of FIG. 14, there is assumed a case where the user currently appreciates a CD at the user's home and the user is impressed by music that the user currently appreciates and gets excited.

A pulse sensor 110s attached to a wrist of the user who currently appreciates music in a living room of the user's home detects a pulse of the user in an excitement state and transmits sensing data to the server 400. In the server 400, the context information acquisition unit 230 analyzes the above sensing data and generates context information including pulse information of the user. Furthermore, the content extraction unit 240 compares and collates the pulse information included in the above context information with metainformation of each piece of content and extracts content matching the above context information. More specifically, the content extraction unit 240 extracts, for example, a musical piece appreciated by the user at the above concert hall, the musical piece having, as metainformation, the number of pulses substantially the same as the number of pulses included in the context information.

According to the fifth example, the server 400 can even associate a state of the user that cannot be easily expressed by words, such as a pulse of the user detected by the sensor 110s, with content as context information indicating the state of the user. Therefore, in a case where content is extracted in the first embodiment, it is possible to also use metainformation based on context information at the time of extracting the content, and therefore it is possible to extract content more suitable for a state of the user.

### 4. Hardware Configuration

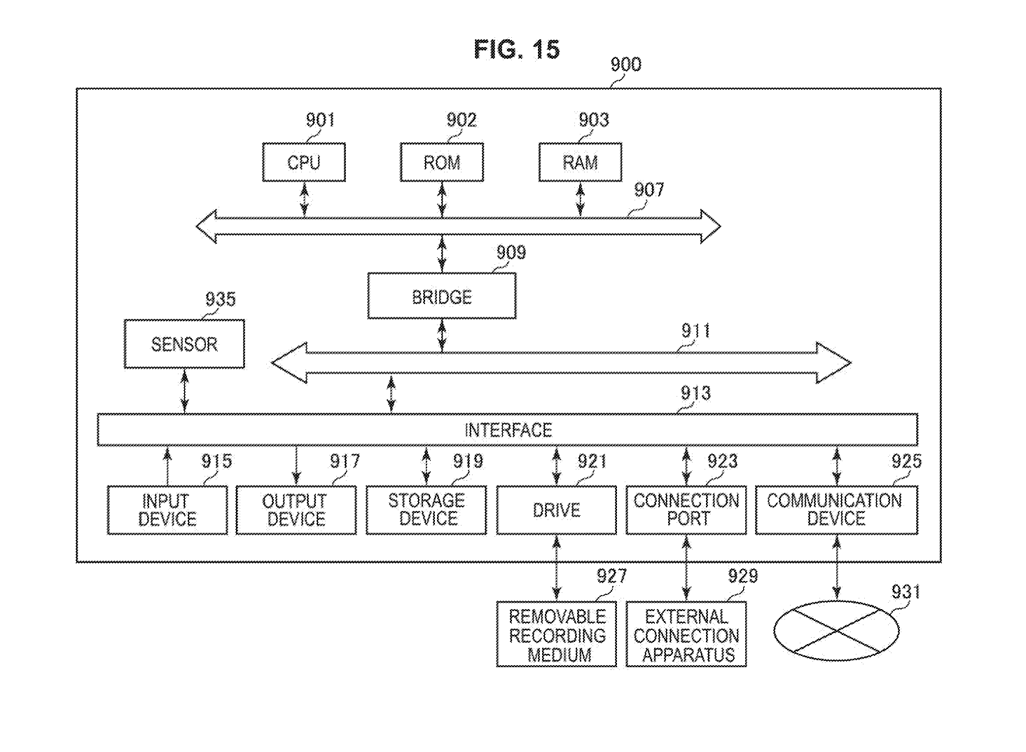

Next, a hardware configuration of the information processing device according to the embodiments of the present disclosure will be described with reference to FIG. 15. FIG. 15 is a block diagram for describing the hardware configuration of the information processing device. An information processing device 900 shown in FIG. 15 can realize, for example, the detection device 100, the server 200, and the terminal device 300 in the above embodiments.

The information processing device 900 includes a central processing unit (CPU) 901, read only memory (ROM) 903, and random access memory (RAM) 905. In addition, the information processing device 900 may include a host bus 907, a bridge 909, an external bus 911, an interface 913, an input device 915, an output device 917, a storage device 919, a drive 921, a connection port 923, and a communication device 925. Moreover, the information processing device 900 may include a sensor 935. The information processing device 900 may include a processing circuit such as a digital signal processor (DSP) alternatively or in addition to the CPU 901.

The CPU 901 functions as an arithmetic processing device and a control device, and controls the overall operation or a part of the operation of the information processing device 900 according to various programs recorded in the ROM 903, the RAM 905, the storage device 919, or a removable recording medium 927. The ROM 903 stores programs, operation parameters, and the like used by the CPU 901. The RAM 905 transiently stores programs used when the CPU 901 is executed, and parameters that change as appropriate when executing such programs. The CPU 901, the ROM 903, and the RAM 905 are connected with each other via the host bus 907 configured from an internal bus such as a CPU bus or the like. The host bus 907 is connected to the external bus 911 such as a Peripheral Component Interconnect/Interface (PCI) bus via the bridge 909.

The input device 915 is a device operated by a user such as a button, a keyboard, a touchscreen, and a mouse. The input device 915 may be a remote control device that uses, for example, infrared radiation and another type of radio waves. Alternatively, the input device 915 may be an external connection apparatus 929 such as a smartphone that corresponds to an operation of the information processing device 900. The input device 915 includes an input control circuit that generates input signals on the basis of information which is input by a user to output the generated input signals to the CPU 901. The user can input various types of data and indicate a processing operation to the information processing device 900 by operating the input device 915.

The output device 917 includes a device that can visually or audibly report acquired information to a user. The output device 917 may be, for example, a display device such as a liquid crystal display (LCD) and an organic electro-luminescence (EL) display, and an audio output device such as a speaker and a headphone. The output device 917 outputs a result obtained through a process performed by the information processing device 900, in the form of text or video such as an image, or sounds such as voice and audio sounds.

The storage device 919 is a device for data storage that is an example of a storage unit of the information processing device 900. The storage device 919 includes, for example, a magnetic storage device such as a hard disk drive (HDD), a semiconductor storage device, or an optical storage device. The storage unit 919 stores therein the programs and various data executed by the CPU 901, and various data acquired from an outside.

The drive 921 is a reader/writer for the removable recording medium 927 such as a magnetic disk, an optical disc, and a semiconductor memory, and built in or externally attached to the information processing device 900. The drive 921 reads out information recorded on the mounted removable recording medium 927, and outputs the information to the RAM 905. The drive 921 writes the record into the mounted removable recording medium 927.

The connection port 923 is a port used to directly connect apparatuses to the information processing device 900. The connection port 923 may be a Universal Serial Bus (USB) port, an IEEE1394 port, or a Small Computer System Interface (SCSI) port, for example. The connection port 923 may also be an RS-232C port, an optical audio terminal, a High-Definition Multimedia Interface (HDMI (registered trademark)) port, and so on. The connection of the external connection device 929 to the connection port 923 makes it possible to exchange various kinds of data between the information processing device 900 and the external connection device 929.

The communication device 925 is a communication interface including, for example, a communication device for connection to a communication network 931. The communication device 925 may be, for example, a communication card for a wired or wireless local area network (LAN), Bluetooth (registered trademark), or a wireless USB (WUSB). The communication device 925 may also be, for example, a router for optical communication, a router for asymmetric digital subscriber line (ADSL), or a modem for various types of communication. For example, the communication device 925 transmits and receives signals in the Internet or transmits signals to and receives signals from another communication device by using a predetermined protocol such as TCP/IP. The communication network 931 to which the communication device 925 connects is a network established through wired or wireless connection. The communication network 931 is, for example, the Internet, a home LAN, infrared communication, or satellite communication.

The sensor 935 includes various sensors such as a motion sensor, a sound sensor, a biosensor, and a position sensor. Further, the sensor 935 may include an imaging device.

The example of the hardware configuration of the information processing device 900 has been described. Each of the structural elements described above may be configured by using a general purpose component or may be configured by hardware specialized for the function of each of the structural elements. The configuration may be changed as necessary in accordance with the state of the art at the time of working of the present disclosure.

### 5. Supplement

The embodiments of the present disclosure descried above may include, for example, an information processing method executed by the described-above information processing device or the described-above system, a program for causing the information processing device to exhibits its function, and a non-transitory tangible medium having the program stored therein. Further, the program may be distributed via a communication network (including a wireless communication) such as the Internet.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An information processing device including:

a context information acquisition unit configured to acquire context information on a state of a user obtained by analyzing information including at least one piece of sensing data regarding the user; and

a content extraction unit configured to extract one or more pieces of content from a content group on the basis of the context information.

(2)

The information processing device according to (1),

in which the at least one piece of sensing data is provided by a motion sensor configured to detect movement of the user.

(3)

The information processing device according to (1) or (2),

in which the at least one piece of sensing data is provided by a sound sensor configured to detect sound generated around the user.

(4)

The information processing device according to any one of (1) to (3),

in which the at least one piece of sensing data is provided by a biosensor configured to detect biological information of the user.

(5)

The information processing device according to any one of (1) to (4),

in which the at least one piece of sensing data is provided by a position sensor configured to detect a position of the user.

(6)

The information processing device according to any one of (1) to (5),

in which the information includes profile information of the user.

(7)

The information processing device according to any one of (1) to (6), further including: an output control unit configured to control output of the one or more pieces of content to the user.

(8)

The information processing device according to (7),

in which the output control unit controls output of the one or more pieces of content on the basis of the context information.

(9)

The information processing device according to (8), further including:

an output unit configured to output the one or more pieces of content.

(10)

The information processing device according to any one of (1) to (9),

in which the content extraction unit calculates a matching degree between the one or more pieces of content and the context information.

(11)

The information processing device according to (10), further including:

an output control unit configured to control output of the one or more pieces of content to the user so that information indicating the one or more pieces of content is arranged and output in accordance with the matching degree.

(12)

The information processing device according to any one of (1) to (11), further including: a metainformation processing unit configured to associate metainformation based on the context information with the one or more pieces of content.

(13)

The information processing device according to any one of (1) to (12), further including:

a sensor configured to provide the at least one piece of sensing data.

(14)

An information processing method including:

acquiring context information on a user obtained by analyzing information including at least one piece of sensing data regarding the user; and

causing a processor to extract one or more pieces of content from a content group on the basis of the context information.

(15)

A program for causing a computer to realize

a function of acquiring context information on a user obtained by analyzing information including at least one piece of sensing data regarding the user, and a function of extracting one or more pieces of content from a content group on the basis of the context information.

### REFERENCE SIGNS LIST

10 system
100 detection device
100a, 100g, 100h, 100i, 100j smartphone
100b, 100m, 100r wristwear
100c imaging device
100d access point
100e, 100f microphone
110 sensing unit
110f, 510 camera
110s pulse sensor
130 transmission unit
200, 400 server
210 reception unit
220 storage
230 context information acquisition unit
240 content extraction unit
250 output control unit
260, 340 transmission unit
300 terminal device
300a, 300b TV
300c projector
300d headphones
330 input unit
350 reception unit
360 output control unit
370 output unit
470 metainformation processing unit
520 content server

1. An information processing device comprising:
a context information acquisition unit configured to acquire context information on a state of a user obtained by analyzing information including at least one piece of sensing data regarding the user; and
a content extraction unit configured to extract one or more pieces of content from a content group on the basis of the context information.

2. The information processing device according to claim 1,
wherein the at least one piece of sensing data is provided by a motion sensor configured to detect movement of the user.

3. The information processing device according to claim 1,
wherein the at least one piece of sensing data is provided by a sound sensor configured to detect sound generated around the user.

4. The information processing device according to claim 1,
wherein the at least one piece of sensing data is provided by a biosensor configured to detect biological information of the user.

5. The information processing device according to claim 1,
wherein the at least one piece of sensing data is provided by a position sensor configured to detect a position of the user.

6. The information processing device according to claim 1,
wherein the information includes profile information of the user.

7. The information processing device according to claim 1, further comprising:
an output control unit configured to control output of the one or more pieces of content to the user.

8. The information processing device according to claim 7,
wherein the output control unit controls output of the one or more pieces of content on the basis of the context information.

9. The information processing device according to claim 8, further comprising:
an output unit configured to output the one or more pieces of content.

10. The information processing device according to claim 1,
wherein the content extraction unit calculates a matching degree between the one or more pieces of content and the context information.

11. The information processing device according to claim 10, further comprising:
an output control unit configured to control output of the one or more pieces of content to the user so that information indicating the one or more pieces of content is arranged and output in accordance with the matching degree.

12. The information processing device according to claim 1, further comprising:
a metainformation processing unit configured to associate metainformation based on the context information with the one or more pieces of content.

13. The information processing device according to claim 1, further comprising:
a sensor configured to provide the at least one piece of sensing data.

14. An information processing method comprising:
acquiring context information on a state of a user obtained by analyzing information including at least one piece of sensing data regarding the user; and
causing a processor to extract one or more pieces of content from a content group on the basis of the context information.

15. A program for causing a computer to realize
a function of acquiring context information on a state of a user obtained by analyzing information including at least one piece of sensing data regarding the user, and
a function of extracting one or more pieces of content from a content group on the basis of the context information.
